(19) United States
(12) Patent Application Publication
(10) Pub. No.: US 2017/0296370 A1
(43) Pub. Date: Oct. 19, 2017

(54) APPARATUS AND METHOD FOR DYNAMIC FOOT SUPPORT

(71) Applicants: A. Jamie Riley, St. Louis, MO (US); Gary J. Schmidt, St. Louis, MO (US)

(72) Inventors: A. Jamie Riley, St. Louis, MO (US); Gary J. Schmidt, St. Louis, MO (US)

(21) Appl. No.: 15/488,178

(22) Filed: Apr. 14, 2017

Related U.S. Application Data
(60) Provisional application No. 62/323,353, filed on Apr. 15, 2016.

(51) Int. Cl. A61F 5/01; A61F 5/14

(52) U.S. Cl. CPC A61F 5/0111; A61F 5/14

(57) ABSTRACT

A banded support system configured to induce a windlass mechanism in a foot includes a forefoot portion configured to enclose at least a portion of a forefoot of the foot, and a band coupled to the forefoot portion. The band includes a first end extending from the forefoot portion in a first orientation, and a second end extending from the forefoot portion in the first orientation. The first end is positioned proximate the second end such that the band is substantially U-shaped. The band further includes a midpoint positioned between the first and second ends in a second orientation perpendicular to the first orientation.

### CROSS REFERENCE TO RELATED APPLICATIONS

This application is a non-provisional and claims priority to U.S. Provisional Patent Application 62/323,353 filed Apr. 15, 2016, which is hereby incorporated by reference in its entirety.

### BACKGROUND

The present invention relates generally to support garments and, more particularly, to an apparatus and method for dynamic foot support, using a banded support system to induce a windlass mechanism in the foot.

The longitudinal arch of the foot, which extends from the heel to the toes, increasingly sags throughout one's lifetime. This sagging may be evidenced by a change in shoe size as the foot elongates over time. Such sagging contributes to foot fatigue, exacerbated arthritis of the foot, and plantar fasciitis. Raising the arch, taking stress off the plantar fascia, or maintaining the arch at an optimal height, improves foot stability and may treat or prevent these issues. At least some known systems attempt to support the arch with orthotics or inserts. The orthotics provide a static upward force on the arch in an attempt to maintain the shape and/or height of the arch. However, such static force is not always successful in providing relief from the above-described problems because the amount of force needed to be therapeutic may be uncomfortable or even intolerable. Some other systems for treating conditions such as plantar fasciitis involve physical therapy systems or methods that may be inaccessible, inconvenient to access, and/or immobile. Moreover, such systems necessarily cannot provide constant, mobile support or relief in one's everyday life.

Therefore, it would be advantageous to provide an apparatus and method that can provide dynamic, comfortable arch support constantly during use and that is accessible and mobile.

### BRIEF SUMMARY

In one aspect, a banded support system configured to induce a windlass mechanism in a foot is provided. The banded support system includes a forefoot portion configured to enclose at least a portion of a forefoot of the foot, and a band coupled to the forefoot portion. The band includes a first end extending from the forefoot portion in a first orientation, and a second end extending from the forefoot portion in the first orientation. The first end is positioned proximate the second end such that the band is substantially U-shaped. The band further includes a midpoint positioned between the first and second ends in a second orientation perpendicular to the first orientation.

In another aspect, a banded support system configured to induce a windlass mechanism in a foot is provided. The banded support system includes a forefoot portion configured to enclose at least a portion of a forefoot of the foot, and a band coupled to the forefoot portion. The band includes a first portion including a first end and a second end. The first portion first end extends from the forefoot portion in a first orientation. The first portion terminates at the first portion second end in a second orientation perpendicular to the first orientation. The band also includes a second portion including a first end and a second end. The second portion first end extends from the forefoot portion in the first orientation. The second portion terminates at the second portion second end in the second orientation. The second portion second end is coupled to the first portion second end.

### BRIEF DESCRIPTION OF THE DRAWINGS

Reference is now made more particularly to the drawings, which illustrate the best presently known mode of carrying out the invention and wherein similar reference characters indicate the same parts throughout the views.

FIG. 1A is a side view of a foot illustrating a windlass mechanism of the foot at rest.

FIG. 1B is a side view of a foot illustrating a windlass mechanism of the foot in a stretched configuration.

FIG. 2 is a top view of a first example embodiment of a banded support system configured to induce the windlass mechanism in the foot, as shown in FIGS. 1A and 1B.

FIG. 3 is a first side view of the banded support system shown in FIG. 2.

FIG. 4 is a second side view of the banded support system shown in FIG. 2.

FIG. 5 is a bottom view of the banded support system shown in FIG. 2.

FIG. 6 is a cut-away perspective view of the banded support system shown in FIG. 2.

FIG. 7 is a cut-away perspective view of a second example embodiment of a banded support system configured to induce the windlass mechanism in the foot, as shown in FIGS. 1A and 1B.

FIG. 8 is a perspective view of a third example embodiment of a banded support system configured to induce the windlass mechanism in the foot, as shown in FIGS. 1A and 1B.

FIG. 9 is a bottom view of the banded support system shown in FIG. 8.

FIG. 10 is a cut-away perspective view of a fourth example embodiment of a banded support system configured to induce the windlass mechanism in the foot, as shown in FIGS. 1A and 1B.

FIG. 11 is an expanded side view of the banded support system shown in FIG. 10.

FIG. 12 is a cut-away perspective view of a fifth example embodiment of a banded support system configured to induce the windlass mechanism in the foot, as shown in FIGS. 1A and 1B.

FIG. 13 is a cut-away perspective view of a sixth example embodiment of a banded support system configured to induce the windlass mechanism in the foot, as shown in FIGS. 1A and 1B.

FIG. 14 is a perspective view of a seventh example embodiment of a banded support system configured to induce the windlass mechanism in the foot, as shown in FIGS. 1A and 1B.

FIG. 15 is a bottom view of the banded support system shown in FIG. 14.

FIG. 16 is a perspective view of an eighth example embodiment of a banded support system configured to induce the windlass mechanism in the foot, as shown in FIGS. 1A and 1B.

Like numerals in the Figures represent the same or functionally similar elements.

### DETAILED DESCRIPTION

In the following detailed description numerous specific details are set forth in order to provide a thorough understanding of the disclosure. However, it will be understood by those skilled in the art that the present disclosure may be practiced without these specific details. For example, the disclosure is not limited in scope to the particular type of industry application depicted in the figures. In other instances, well-known methods, procedures, and components have not been described in detail so as not to obscure the present disclosure.

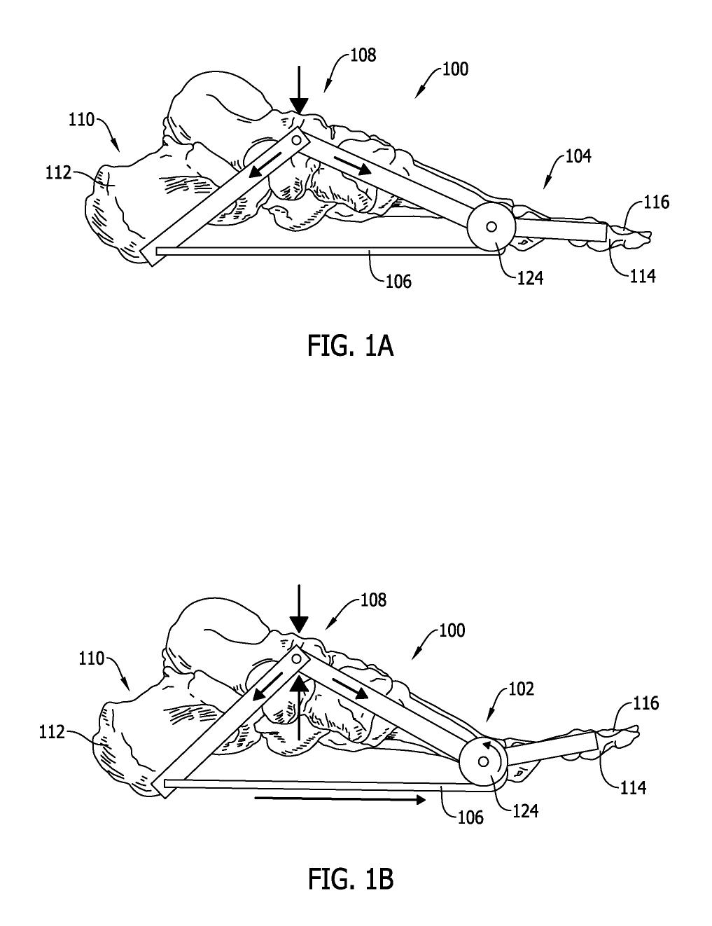

FIGS. 1A and 1B are side views of a foot 100 illustrating a windlass mechanism 102 of foot 100. Foot 100 includes, in pertinent part, a forefoot 104, a plantar fascia 106, and arch or mid-foot 108, and a heel 110 including a calcaneus 112. Forefoot 104 includes a great or first toe 114, a second toe 116, a third toe (not shown), a fourth toe (not shown), a fifth toe (not shown), and a metatarsal head 124 between each toe and mid-foot 108 (e.g., the “ball” of foot 100). Windlass mechanism 102 includes flexing of great toe 114, which pulls plantar fascia 106 about metatarsal head 124. Such a pull on plantar fascia 106 in turn induces a forward pull on calcaneus 112, such that plantar fascia 106 acts as a “tie rod” spanning mid-foot 108 from metatarsal head 124 to calcaneus 112. As a result, a distance between each metatarsal head 124 and calcaneus 112 is decreased. Accordingly, dynamic implementation or inducement of windlass mechanism 102 on foot 100, as illustrated, increases a height of mid-foot or arch 108. Moreover, windlass mechanism 102 “packs in” the bones of mid-foot 108 and facilitates locking of mid-foot 108, such that forefoot 104 acts as a more rigid lever for propulsion (e.g., during walking, running, etc.). Directional terms may be understood as follows: with respect to the view of FIGS. 1A and 1B, “medial” indicates generally “outward” from the illustration (i.e., extending toward the middle of foot 100), and “lateral” indicates generally “inward” into the illustration (i.e., extending away from the middle of foot 100).

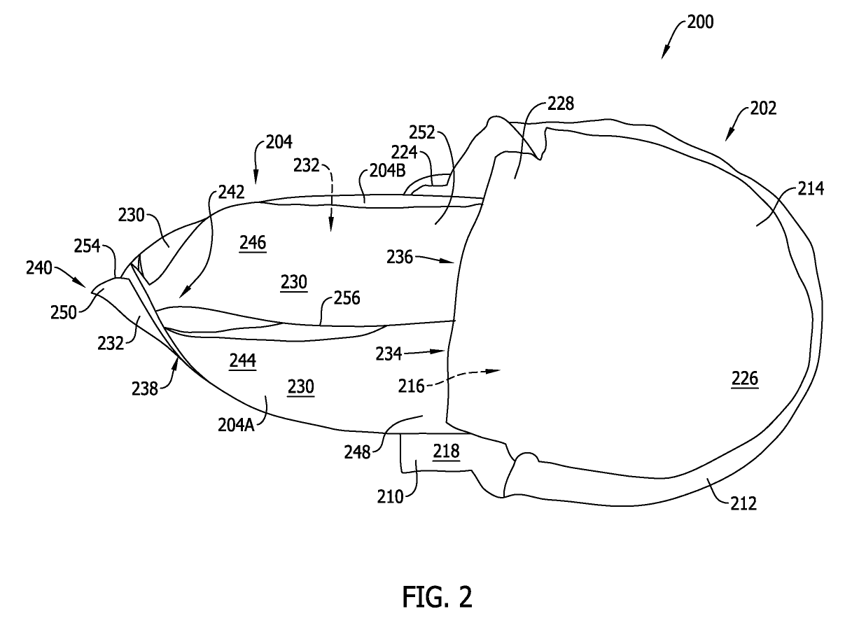

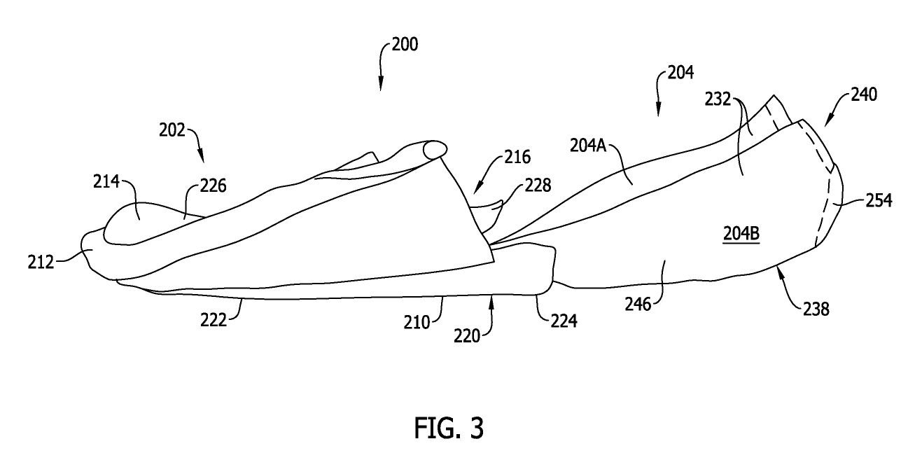

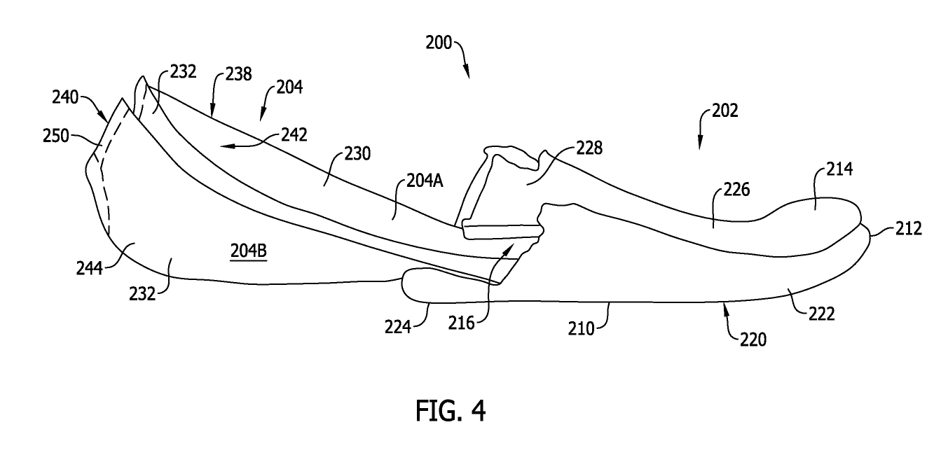

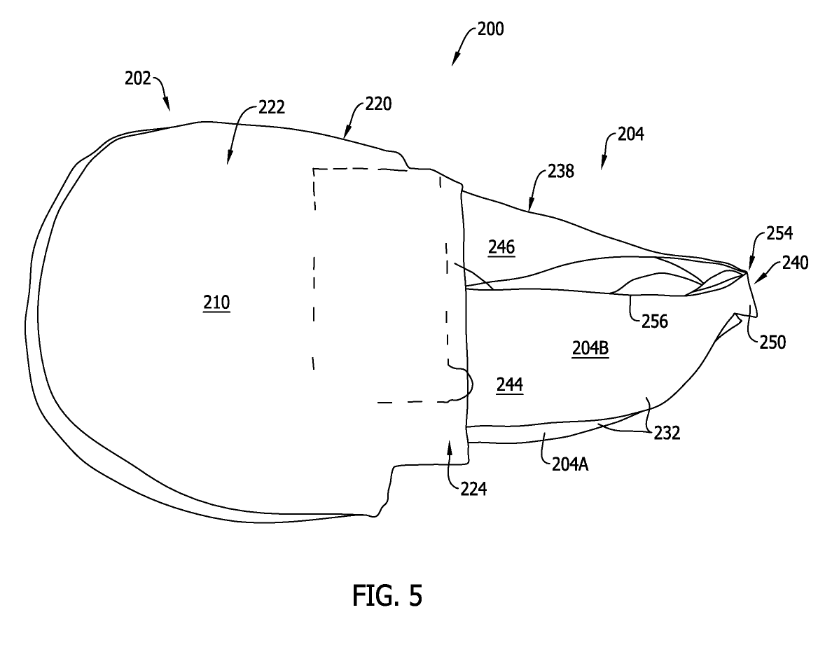

FIGS. 2-6 depict various views of a banded support system 200 configured to dynamically induce windlass mechanism 102 in foot 100 (both shown in FIG. 1). More particularly, banded support system 200 is a mobile apparatus configured to induce windlass mechanism 102 substantially constantly during wear or use, providing resting compression of foot 100 to lift mid-foot 108 and shorten plantar fascia 106. “Mobile,” as used herein, refers to an apparatus configured to be with or on a person regardless of location and/or position, in contrast to an apparatus limited in application to particular locations and/or positions. “Resting compression,” as used herein, refers to an application of compression without necessitating action or movement of the person wearing banded support system 200. For example, “resting compression” occurs while a user of banded support system 200 is sitting (i.e., no active motion applied to foot 100 by the user).

In the illustrated embodiment, banded support system 200 includes a forefoot portion 202 and a band 204. In one embodiment, forefoot portion 202 and band 204 are integrally formed as one unitary piece. In such embodiments, band 204 is integrated into forefoot portion 202 during fabrication thereof, for example, is woven into the weave of forefoot portion 202 or are integrally coupled to the weave of forefoot portion 202. Alternatively, forefoot portion 202 and band 204 are distinct, separate components coupled together.

Forefoot portion 202 is configured to surround and/or enclose at least a portion of forefoot 104 of foot 100, in the manner of a typical sock, when banded support system 200 is placed on foot 100. Forefoot portion 202 may enclose one toe (e.g., first or great toe 114), less than all of the toes (e.g., great toe 114 and second toe 116), or all of the toes, corresponding respective metatarsal head(s) 124, and/or at least a portion of mid-foot 108 (all shown in FIGS. 1A and 1B).

Forefoot portion 202 includes a bottom panel 210, an intermediate panel 212, and a top panel 214. Bottom panel 210, intermediate panel 212, and top panel 214 are separate components coupled together. Alternatively, bottom panel 210, intermediate panel 212, and/or top panel 214 are integrally formed, for example, woven together. Bottom panel 210, intermediate panel 212, and top panel 214 form a cavity 216 for receiving, surrounding, and/or enclosing the portion of forefoot 104, described above, therein. In the example embodiment, top panel 214 is fabricated from a non-elastic material, bottom panel 210 is fabricated from a semi-elastic material, and intermediate panel 212 is fabricated from an elastic material. Alternatively, bottom panel 210 is fabricated from a non-elastic material. “Non-elastic,” “semi-elastic,” and “elastic” are used herein as relative terms. In other words, an “elastic” material is more elastic and/or exhibits more elastic properties than a “semi-elastic” material. A semi-elastic material is more elastic and/or exhibits more elastic properties than a “non-elastic” material. Additionally, “non-elastic” refers generally to materials that have little or no “give”, “stretch,”, or “expansion” such that they retain their size even under tensile or expansion forces. Elastic materials include, for example, elastic nylon. Semi-elastic materials include, for example, neoprene. Non-elastic materials include, for example, non-elastic nylon. Moreover, at least one of bottom panel 210, intermediate panel 212, and/or top panel 214 is fabricated from a material with a relatively high coefficient of static friction on at least one face thereof, or a “tacky” material. For example, bottom panel 210 may be fabricated from a tacky material with a tacky top face 218, such that forefoot 104 experiences less slippage or movement within forefoot portion 202. Additionally, bottom panel 210 may be fabricated from a cushioning material having a thickness and softness such that a cushioning of forefoot 204 is provided. In one embodiment, bottom panel 210 is fabricated from a semi-elastic, tacky, cushioning material, such as a tacky neoprene.

In the example embodiment, the relative elasticity between the panels 210, 212, 214 of forefoot portion 202 causes or encourages a user's toes to be urged forward in forefoot portion 204. Accordingly, one or more of the first through fifth toes are anchored or retained in intermediate panel 212, the most elastic panel. This retention of the toes helps to ensure that the toes remain in the proper placement for activation of windlass mechanism 102 without the need for internal structure in forefoot portion 102, which may be less comfortable for some users. Retention of the toes in intermediate panel 212 may additionally facilitate inducement of dorsiflexion of great toe 114.

Bottom panel 210 includes an elliptical portion 222 with a substantially semi-elliptical shape, corresponding to an approximate outline of forefoot 104. In some embodiment, bottom panel 210 further includes an extension portion 224 extending from elliptical portion 222. Extension portion 224 provides additional surface area of bottom panel 210 for coupling band 204 to bottom panel 210 and/or for increased user comfort. Extension portion 224, in the example embodiment, has a reduced width to complement the narrowing shape of midfoot 108 (shown in FIGS. 1A and 1B). Top panel 214 has a shape that is substantially complementary to the shape of bottom panel 210. More particularly, top panel 214 includes an elliptical portion 226 with a substantially semi-elliptical shape, corresponding to an approximate outline of forefoot 104. Top panel 214 further includes an extension portion 228. Alternatively, top panel 214 does not include an extension portion. In the example embodiment, intermediate panel 212 has a substantially rectangular shape. Intermediate panel 212 is coupled to a perimeter of elliptical portion 222 of bottom panel 210 and a perimeter of elliptical portion 226 of top panel 214. Intermediate panel 212 forms an intermediate wall or interface extending from bottom panel 210 to top panel 214.

Band 204 is coupled to forefoot portion 202. In the example embodiment, band 204 is substantially U-shaped and is configured to engage heel 110 (shown in FIGS. 1A and 1B) of foot 100 when banded support system 200 is placed on foot 100. More particularly, band 204 is configured to curve around heel 110 from a medial side thereof to a lateral side thereof to fully engage heel 110 when banded support system 200 is worn. As used herein, “U-shaped” refers generally to an approximate U-shape with two “legs” and a rounded or curved midpoint between the two legs. “Substantially U-shaped” or “U-shaped” should be also understood to include shapes with less rounded midpoint, such as an approximate V-shape. In one embodiment, band 204 includes a single band 204. In another embodiment, band 204 includes two bands 204, for example, to provide additional tensile or elastic force about heel 110 to ensure retention of band 204 around heel 110. It should be understood that where general reference to “band 204” is made herein, such description may refer to a one-band embodiment and/or to a two-band embodiment.

Band 204 is fabricated from elastic material(s) exhibiting sufficient elasticity to induce windlass mechanism 102 in foot 100. Accordingly, different banded support systems 200 may include bands 204 with different elastic characteristics, such as a stronger elastic band 204 (i.e., having a higher spring constant) for users requiring more elastic force to induce windlass mechanism 102, or a less elastic band 204 (i.e., having a lower spring constant) for users requiring less force to induce windlass mechanism 102. Therefore, band 204 provides a high level of control of the elastic force imposed on foot 100 while maintaining the comfort of the user of banded support system 200.

Band 204 includes a first surface 230, also referred to as a “foot-facing” surface, and an opposing second surface 232. Band 204 further includes a first end 234 and a second end 236. In the example embodiment, first end 234 and second end 236 are coupled to forefoot portion 202. More specifically, first end 234 and second end 236 are coupled to and extend from bottom panel 210 of forefoot portion 202. First end 234 and second end 236 are integrally formed. Alternatively, first end 234 and second end 236 are separate, distinct ends of band 204. First end 234 and second end 236 are positioned proximate one another, in the example embodiment. The relative positions of first end 234 and second end 236 define the U-shape of band 204. Additionally, first end 234 and second end 236 are coupled to forefoot portion 202 in a first, “horizontal” orientation. In the first orientation, first surface 230 of first and second ends 234, 236 is facing “upwards” or towards foot 100 (when worn), and second surface 232 is coupled against top face 218 of bottom panel 210. In an alternative embodiment, in the first orientation, first surface 230 of first and second ends 234, 236 is facing “upwards” and is coupled against a bottom face 220 of bottom panel 210, and second surface 232 of first and second ends 234, 236 is facing “downwards.”

Band 204 further includes a body 238 extending between first end 234 and second end 236. Body 238 includes a midpoint 240 between first end 234 and second end 236. In the illustrated embodiment, midpoint 240 is in a second, “vertical” orientation that is substantially perpendicular to the first orientation. Midpoint 240 forms a U-shaped heel portion 242 configured to receive heel 110. Extending body 238 to form heel portion provides an anchor for primary band 204 and also facilitates establishing an optimal or predetermined amount of force across body 238 of band 204. In other words, extending body 238 tighter through heel portion 242 induces a greater amount of force across body 238 than extending body 238 more loosely through heel portion 242. Body 238 also includes a first portion 244 extending between first end 234 and midpoint 240 and a second portion 246 extending between second end 236 and midpoint 240. First portion 244 and second portion 246 are accordingly substantially equal in length. First portion 244 includes a first end 248 corresponding to first end 234 of band 204 and a second end 250 positioned at midpoint 240. Second portion 246 includes a first end 252 corresponding to second end 236 of band 204 and a second end 254 positioned at midpoint 240. As such, first portion first end 248 is positioned adjacent second portion first end 252, and first portion second end 250 is positioned adjacent second portion second end 254. In one embodiment, second end 250 of first portion 244 and second end 254 of second portion 246 are separate and distinct. Second end 250 of first portion 244 and second end 254 of second portion 246 are coupled together in the second orientation at midpoint 240, thereby defining midpoint 240 at the intersection thereof. Alternatively, second end 250 of first portion 244 and second end 254 of second portion 246 are integrally formed. In the illustrated embodiment, first portion 244 is substantially parallel with and/or complementary to second portion 246, such that first portion 244 and second portion 246 transition from the first orientation at first ends 248, 252 to the second orientation at second ends 250, 254 in substantially the same manner.

In one embodiment, first and second portions 244, 246 are formed by cutting or otherwise dividing one unitary band 204 along a cutline 256. In one embodiment, cutline 256 extends through band 204 between first end 234 and second end 236, such that first ends 248, 252 of first and second portions 244, 246, respectively, are separated from each other. In another embodiment, cutline 256 extends through band 204 at midpoint 240 of band 204, such that second ends 250, 254 of first and second portions 244, 246, respectively, are separated from each other. In yet another embodiment, cutline 256 extends throughout band 204, such that first portion 244 and second portion 246 are entirely separate. Alternatively, band 204 is formed from distinct first portion 244 and second portion 246. In other words, two separate pieces of material are coupled together and to forefoot portion 202 to form band 204.

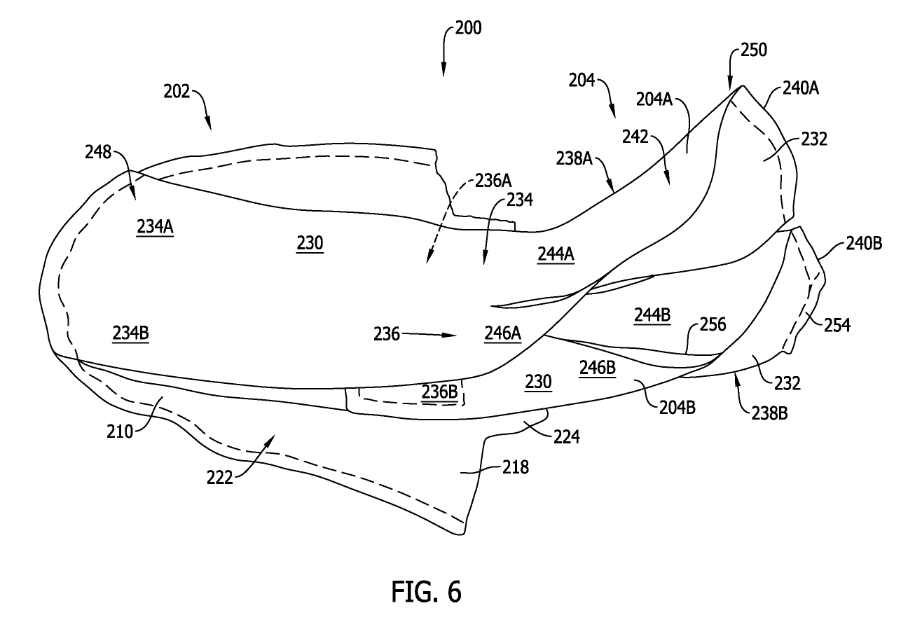

In the illustrated embodiment, band 204 includes a first band 204A and a second band 204B (see FIG. 6). As described above, first band 204A includes respective first and second ends 234A, 236A and a respective midpoint 240A. Second band 204B includes respective first and second ends 234B, 236B and a respective midpoint 240B. Midpoints 240A and 240B are adjacent one another without a substantial gap therebetween. However, as shown in FIG. 6, first end 234A and second end 236A of first band 204A extend longitudinally across the entirety of elliptical portion 222 of bottom panel 210. First end 234A and second end 236A of first band 204A are coupled to bottom panel 210 adjacent a perimeter thereof. On the other hand, first end 234B and second end 236B of second band 204B are coupled to bottom panel 210 at or adjacent to extension portion 224 of bottom panel 210. In other words, first and second bands 204A, 204B have different lengths of bodies 238A, 238B, respectively. Having bands 204A, 204B of different lengths may induce different levels of longitudinal tensile force on foot 100 across each respect band 204A, 204B (e.g., less tensile force longitudinally across first band 204A and more tensile force longitudinally across second band 204B). This variation in tensile force across bands 204A, 204B may provide certain therapeutic benefits. Additionally, the particular location of the coupling of first end(s) 234A and/or 234B and second end(s) 236A and/or 236B of band(s) 204A and/or 204B may facilitate providing maximum compression to arch 108 and plantar fascia 106. It should be understood that although first and second bands 204A, 204B are shown coupled to top face 218 of bottom panel 210, in an alternative embodiment, one or more of first and/or second bands 204A, 204B is coupled to bottom face 220 of bottom panel 210.

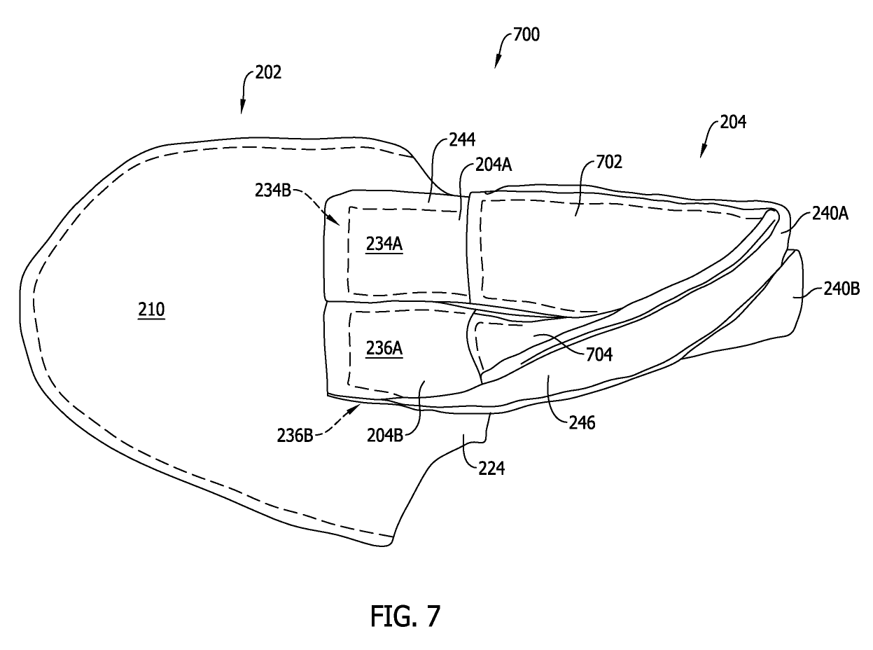

FIG. 7 is a cut-away perspective view of a second example embodiment of a banded support system 700 configured to induce windlass mechanism 102 in foot 100 (both shown in FIGS. 1A and 1B). The view of FIG. 7 is “cut-away” in the sense that a top panel (e.g., top panel 214, shown in FIG. 2-5) has been removed or “cut-away” from view to reveal the interior of forefoot portion 202. In the illustrated embodiment, band 204 includes first band 204A and second band 204B (as shown in FIGS. 2-6). However, first band 204A and second band 204B have substantially equal lengths. First end 234A and second end 236A of first band 204A are coupled to bottom panel 210 at or adjacent to extension portion 224. Similarly, first end 234B and second end 236B of second band 204B are coupled to bottom panel 210 at or adjacent to extension portion 224. Accordingly, first and second bands 204A, 204B may “work together” to exert a specific amount of longitudinal tensile for on foot 100 for a particular therapeutic benefit. Moreover, in the illustrated embodiment, midpoint 240A, 240B of both first and second bands 204A, 204B, respectively, are integrally formed. In other words, for both first and second bands 204A, 204B, second end 250 of first portion 244A/244B and second end 254 of second portion 246A/246B are integrally connected at midpoints 240A, 240B. In addition, first end 234A and second end 236A of first band 204A are separated from one another, and first end 234B and second end 236B of second band 204B are separated from one another. First band 204A is therefore one integral band that originates at first end 234A and extends continuously through midpoint 240A to second end 236A. Similarly, second band 204B is one integral band that originates at first end 234B and extends continuously through midpoint 240B to second end 236B.

Further, band 204 (more particularly first band 204A, although continued reference will be made to general, collective band 204) includes a first band cushioning member 702 coupled to first portion 244 and a second band cushioning member 704 coupled to second portion 246. In the example embodiment, first and second band cushioning members 702, 704 are coupled to first or foot-facing surface 230 of band 204. Band cushioning members 702, 704 are fabricated from a material having a suitable thickness and softness to provide a cushioning effect to foot 100 of a user of banded support system 700 when banded support system 700 is worn. Specifically, band cushioning members 702, 704 are configured to provide a cushioning effect between foot 100 and band 204 (e.g., to avoid blisters and/or chafing). Band cushioning members 702, 704 are fabricated from, for example, neoprene. Although two separate band cushioning members 702, 704 are illustrated, it should be understood that in an alternative embodiment, band 204 has a single, continuous band cushioning member coupled thereto that extends substantially the entire length of first and second band cushioning members 702, 704 as shown.

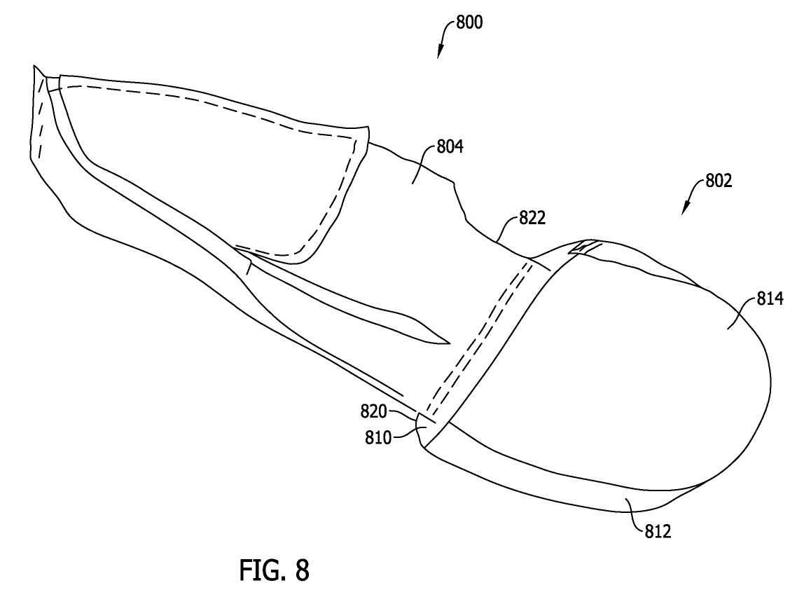

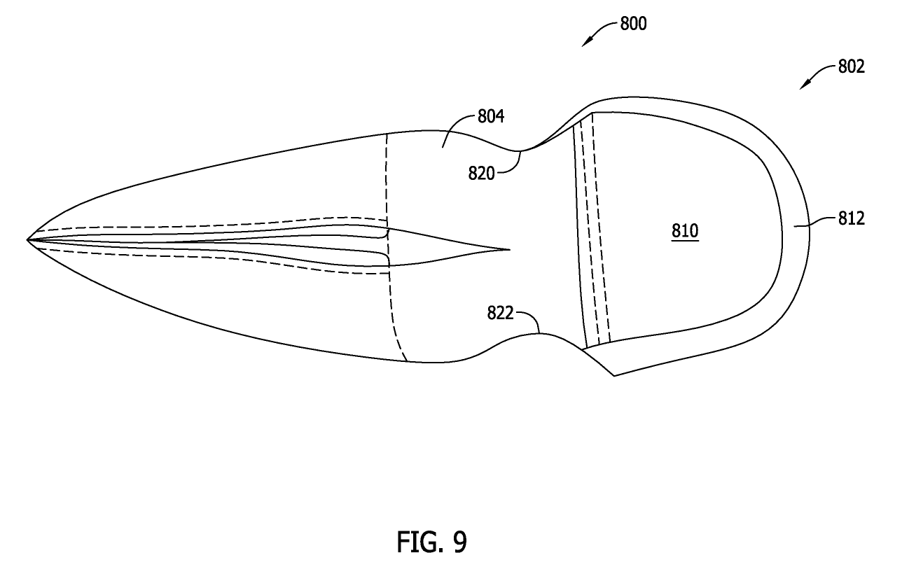

FIGS. 8 and 9 illustrate a third example embodiment of a banded support system 800 configured to induce windlass mechanism 102 in foot 100 (both shown in FIGS. 1A and 1B). More particularly, FIG. 8 is a perspective view of banded support system 800, and FIG. 9 is a bottom view of banded support system 800. In the illustrated embodiment, a forefoot portion 802 of banded support system 800 is much narrower than forefoot portion 202 of banded support system 200 (both shown in FIGS. 2-6). Forefoot portion 802 is configured to receive less than all of a user's toes, such as only great toe 114 or only great toe 114 and second toe 116 (both shown in FIGS. 1A and 1B). Forefoot portion 802 includes a bottom panel 810, intermediate panel 812, and top panel 814 that are similar in structure and function to corresponding bottom panel 210, intermediate panel 212, and top panel 214 (all shown in FIGS. 2-6) of forefoot portion 202, but that are dimensionally reduced. Moreover, in the illustrated embodiment, a band 804 includes only a single band 804. In some embodiments, single band 804 may be more elastic (e.g., have a higher spring constant) than, for example, first band 204A and second band 204B (shown in FIGS. 2-6) such that single band 804 may exert a suitable amount of longitudinal force to induce windlass mechanism 102 in foot 100. In addition, band 804 includes cutouts 820, 822 configured to accommodate the reduced width of forefoot portion 802.

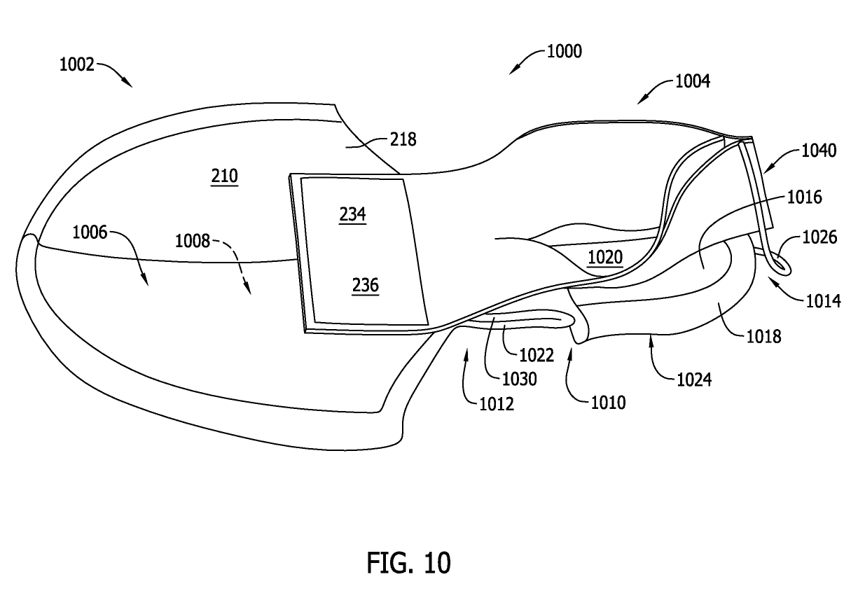

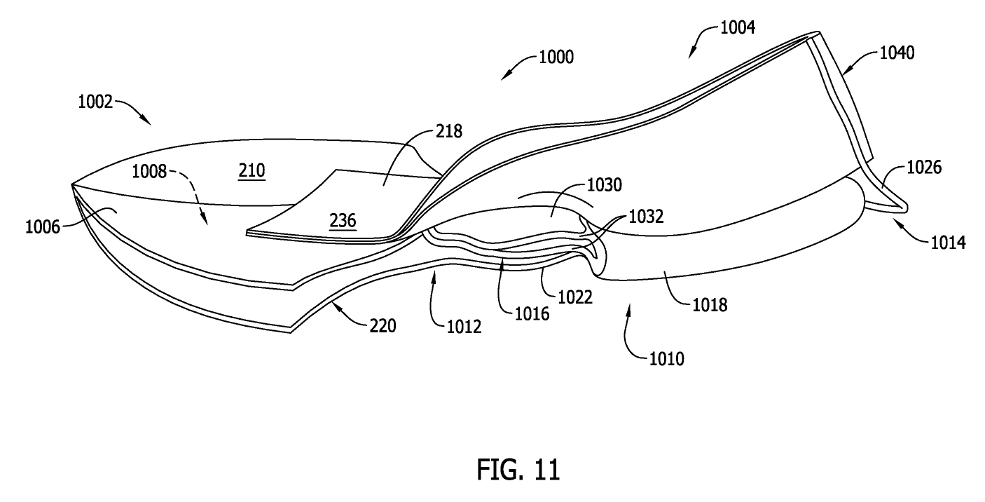

FIGS. 10 and 11 illustrate a fourth example embodiment of a banded support system 1000 configured to induce windlass mechanism 102 in foot 100 (both shown in FIGS. 1A and 1B). More particularly, FIG. 10 is a cut-away perspective view of banded support system 1000, and FIG. 11 is an expanded side view of banded support system 1000. The view of FIG. 10 is “cut-away” in the sense that a top panel (e.g., top panel 214, shown in FIGS. 2-5) has been removed or “cut-away” from view to reveal the interior of a forefoot portion 1002 of banded support system 1000. Banded support system 1000 includes forefoot portion 1002 and a band 1004. In the illustrated embodiment, forefoot portion 1002 further includes a forefoot cushioning member 1006. More specifically, forefoot cushioning member 1006 is coupled to bottom panel 210 of forefoot portion 1002. In the illustrated embodiment, forefoot cushioning member 1006 is coupled to top surface 218 of bottom panel 210, although in an alternative embodiment, forefoot cushioning member 1006 is coupled to bottom surface 220 of bottom panel 210. Moreover, in the illustrated embodiment, forefoot cushioning member 1006 is disposed between second end 236 of band 1004 and bottom panel 210. In an alternative embodiment, forefoot cushioning member 1006 is positioned on top of second end 236 of band 204.

Additionally, forefoot cushioning member 1006 is coupled to a medial region 1008 of bottom panel 210, corresponding to a medial side of forefoot 104 of foot 100. Forefoot cushioning member 1006 is fabricated from a material having a suitable thickness and softness to provide a cushioning effect to foot 100 of a user of banded support system 1000 when banded support system 1000 is worn. Specifically, forefoot cushioning member 1006 is configured to provide a cushioning effect between a ball of foot 100 and bottom panel 210. Forefoot cushioning member 1006 is further configured to facilitate medial-to-lateral alignment of forefoot 104 by providing additional height to the medial side of forefoot 104. For example, forefoot cushioning member 1006 may add about 4 mm of height (taken from a bottom surface of forefoot cushioning member 1006 to a top surface of forefoot cushioning member) to medial region 1008 of bottom panel 210. Forefoot cushioning member 1006 is fabricated from, for example, neoprene. In one embodiment, forefoot cushioning member 1006 has a shape substantially the same as medial region 1008 of bottom panel 210. In an alternative embodiment, forefoot cushioning member 1006 has any other shape suitable to provide cushioning and/or alignment.

In addition, banded support system 1000 includes a hindfoot portion 1010. Hindfoot portion 1010 includes a first end 1012 and a distal second end 1014. Hindfoot portion 1010 extends from forefoot portion 1002 at first end 1012. Second end 1014 corresponds to heel 110 (specifically, a bottom face thereof) of foot 100. Hindfoot portion 1010 also includes a bottom panel 1016 and a flange 1018. Bottom panel 1016 includes a first or “foot-facing” surface 1020 and an opposing second surface 1022.

First end 1012 is coupled to bottom panel 210 of forefoot portion 1002. Specifically, bottom panel 1016 of hindfoot portion 1010 is coupled to bottom panel 210 of forefoot portion 1002 at first end 1012. In one embodiment, bottom panel 1016 of hindfoot portion 1010 and bottom panel 210 of forefoot portion 1002 are separate components coupled together at first end 1012. In another embodiment, bottom panel 1016 of hindfoot portion 1010 and bottom panel 210 of forefoot portion 1002 are integrally formed as one component or piece of material. Bottom panel 1016 of hindfoot portion 1010 is fabricated from the same material as bottom panel 210 of forefoot portion 1002 (e.g., a semi-elastic neoprene). In an alternative embodiment, bottom panel 1016 of hindfoot portion 1010 is fabricated from a different material than bottom panel 210 of forefoot portion 1002.

In the example embodiment, bottom panel 1016 extends from first end 1012 to second end 1014. Bottom panel 1016 has a shape substantially corresponding to a shape of foot 100, specifically a bottom surface thereof. Flange 1018 extends around a perimeter 1024 of second end 1014. In one embodiment, flange 1018 includes a separate component coupled to bottom panel 1016. In an alternative embodiment, flange 1018 is integrally formed with bottom panel 1016. Flange 1018 acts as a barrier or support to keep heel 110 from slipping off of bottom panel 1016 when banded support system 1000 is worn. Flange 1018 is fabricated from a material having sufficient thickness and/or stiffness to provide such support. For example, flange 1018 is fabricated from a folded-over neoprene.

Additionally, hindfoot member 1010 is coupled to band 1004 to ensure placement of both band 1004 and second end 1014 of hindfoot member 1010 when banded support system 1000 is worn. In the illustrated embodiment, second end 1014 of hindfoot member 1010 is coupled to midpoint 1040 of band 1004 via an elastic retention band 1026. In other embodiments, hindfoot member 1010 is coupled to band 1004 using an alternative coupling method (e.g., a differently positioned retention band).

Hindfoot portion 1010 further includes an arch support member 1030. Arch support member 1030 is coupled to bottom panel 1016 of hindfoot portion 1010 at a location corresponding to arch or midfoot 108 (shown in FIGS. 1A and 1B) of foot 100. In the illustrated embodiment, arch support member 1030 is coupled to first or foot-facing surface 1020 of bottom panel 1016. In an alternative embodiment, arch support member 1030 is coupled to second face 1022 of bottom panel 1016. Arch support member 1030 is configured to provide support to arch 108 when banded support system 1000 is worn. Arch support member 1030 is fabricated from a material suitable to provide such support. For example, arch support member 1030 is fabricated from at least one layer 1032 of neoprene. Arch support member 1030 may include one layer 1032 or more than one layer 1032 of fabric to adjust a height of arch support member 1030, according to the arch support needs of the user of banded support system 1000.

In the illustrated embodiment, forefoot cushioning member 1006 and arch support member 1030 together provide improved alignment of foot 100 when banded support system 1000 is worn, compared to alternative embodiments of banded support system 1000. Specifically, forefoot cushioning member 1006 and arch support member 1030 provide alignment for “flat” feet 100, wherein arch 108 is relatively low.

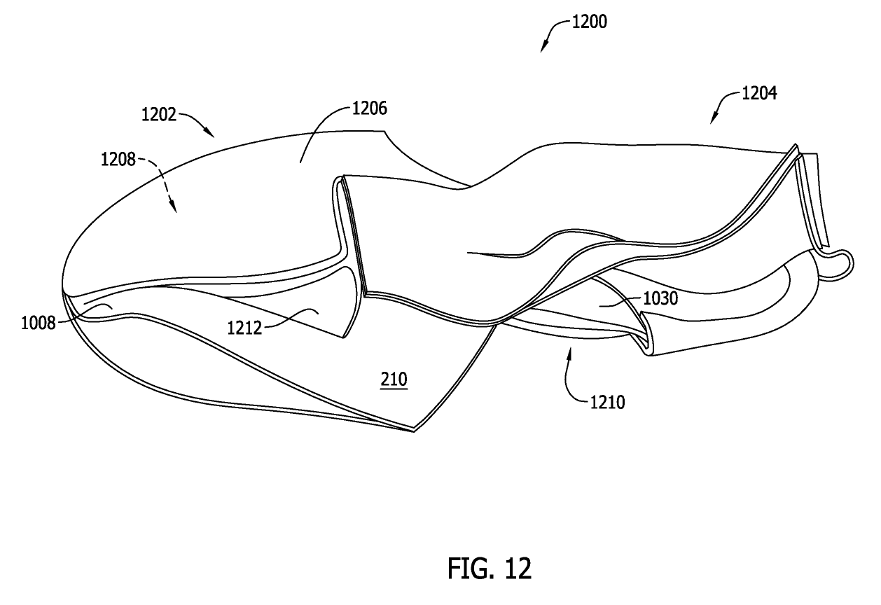

FIG. 12 is a cut-away perspective view of a fifth example embodiment of a banded support system 1200 configured to induce windlass mechanism 102 in foot 100 (both shown in FIGS. 1A and 1B). The view of FIG. 12 is “cut-away” in the sense that a top panel (e.g., top panel 214, shown in FIGS. 2-5) has been removed or “cut-away” from view to reveal the interior of a forefoot portion 1202 of banded support system 1200. Banded support system 1200 includes a forefoot portion 1202, band 1204, and hindfoot portion 1210 substantially similar to forefoot portion 1002, band 1004, and hindfoot portion 1010 (all shown in FIGS. 10 and 11), respectively.

Banded support system 1200 includes a forefoot cushioning member 1206 coupled to a lateral region 1208 of bottom panel 210 of forefoot portion 1202, corresponding to a lateral side of forefoot 104 of foot 100. Similar to forefoot cushioning member 1006 (also shown in FIGS. 10 and 11), forefoot cushioning member 1206 is fabricated from a material having a suitable thickness and softness to provide a cushioning effect to foot 100 of a user of banded support system 1200 when banded support system 1200 is worn. Specifically, forefoot cushioning member 1206 is configured to provide a cushioning effect between a ball of foot 100 and bottom panel 210. Forefoot cushioning member 1206 is further configured to facilitate medial-to-lateral alignment of forefoot 104 by providing additional height to the lateral side of forefoot 104. For example, forefoot cushioning member 1206 may add about 4 mm of height (taken from a bottom surface of forefoot cushioning member 1206 to a top surface of forefoot cushioning member) to lateral region 1208 of bottom panel 210. Forefoot cushioning member 1206 is fabricated from, for example, neoprene. In one embodiment, forefoot cushioning member 1206 has a shape substantially the same as lateral region 1208 of bottom panel 210. In an alternative embodiment, forefoot cushioning member 1206 has any other shape suitable to provide cushioning and/or alignment.

Banded support system 1200 further includes a cutout 1212 in medial region 1008 (shown in FIGS. 10 and 11) of bottom panel 210. Cutout 1212 is configured to receive at least a portion of the medial side of forefoot 104 therein, such that that portion of forefoot 104 is in a lower position that the lateral side of forefoot 104. Cutout 1212, forefoot cushioning member 1206, and arch support member 1030 (also shown in FIGS. 10 and 11) together provide improved alignment of foot 100 when banded support system 1200 is worn, compared to alternative embodiments of banded support system 1200. Specifically, cutout 1212, forefoot cushioning member 1206, and arch support member 1030 provide alignment for “high arch” feet 100, wherein arch 108 is relatively high.

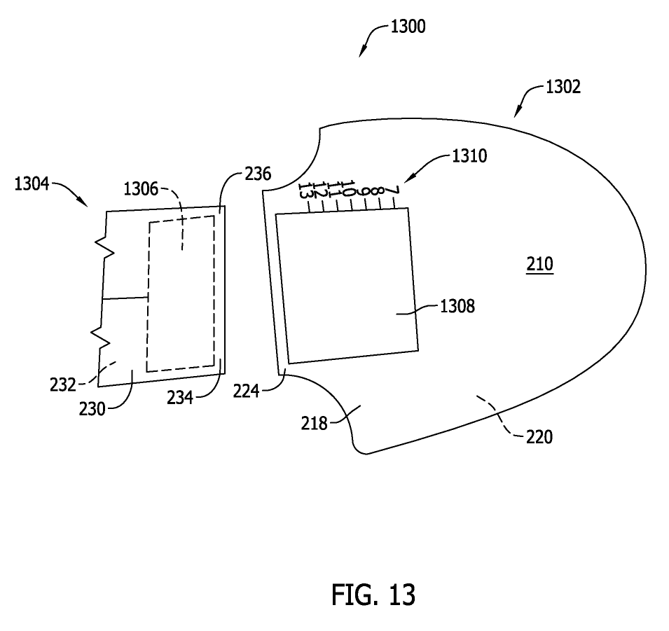

FIG. 13 is a cut-away perspective view of a sixth example embodiment of a banded support system 1300 configured to induce windlass mechanism 102 in foot 100 (both shown in FIGS. 1A and 1B). The view of FIG. 13 is “cut-away” in the sense that a top panel (e.g., top panel 214, shown in FIGS. 2-5) has been removed or “cut-away” from view to reveal the interior of a forefoot portion 1302 of banded support system 1300.

In the illustrated embodiment, banded support system 1300 includes band 1304. More specifically, band 1304 is a removable band 1304. A band-surface joining member 1306 is coupled to second surface 232 (shown in FIGS. 2-6) of band 1304, and a panel-surface joining member 1308 is coupled to top face 218 of bottom panel 210 (both shown in FIGS. 2-6). Alternatively, band-surface joining member 1306 is coupled to first surface 230 (also shown in FIGS. 2-6) of band 1304, and panel-surface joining member 1308 is coupled to bottom surface 220 (also shown in FIGS. 2-6) of bottom panel 210. In one embodiment, band-surface joining member 1306 is coupled to one of first end 234 and second end 236 (both shown in FIGS. 2-6) of band 1304. In another embodiment, band-surface joining member 1306 is coupled to both of first end 234 and second end. Panel-surface joining member 1308 is coupled to and/or adjacent to extension portion 224 of bottom panel 210. Band-surface joining member 1306 is configured to couple to panel-surface joining member 1308 to couple band 1304 to bottom panel 210. In one embodiment, band-surface joining member 1306 and panel-surface joining member 1308 are complementary hook-and-loop fasteners (e.g., one of joining member 1306 and 1308 is the “hook” member and the other is the “loop” member). Alternatively, band-surface joining member 1306 and panel-surface joining member 1308 may be any other complementary fasteners. In another embodiment, banded support system 1300 includes only one joining member on only one of band 1304 and bottom panel 210 (e.g., an adhesive region).

In addition, bottom panel 210 and/or panel-surface joining member 1308 includes size references 1310. Size references 1310 are configured to indicate to a user of banded support system 1300 where to couple band-surface joining member 1306 to panel-surface joining member 1308 to adjust the amount of force exerted by band 1304 according to foot and/or shoe size. For example, to ensure that band 1304 exerts an appropriate amount of force on a foot 100 of shoe size 8, the user of banded support system 1300 would line up band-surface joining member 1306 along panel-surface joining member 1308 at a sizing reference 1310 associated with the shoe size 8 (e.g., a numeral “8” printed on bottom panel 210 and/or panel-surface joining member 1308).

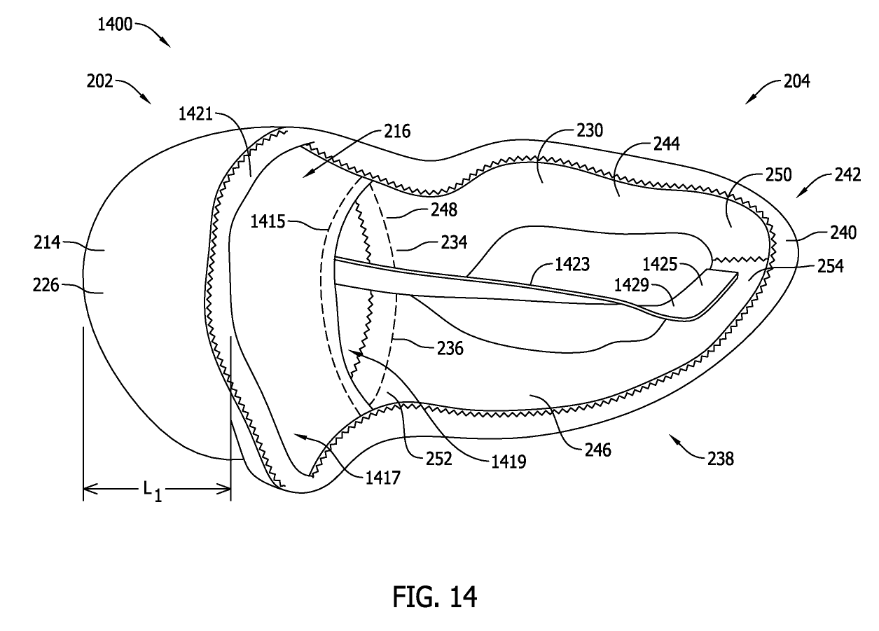

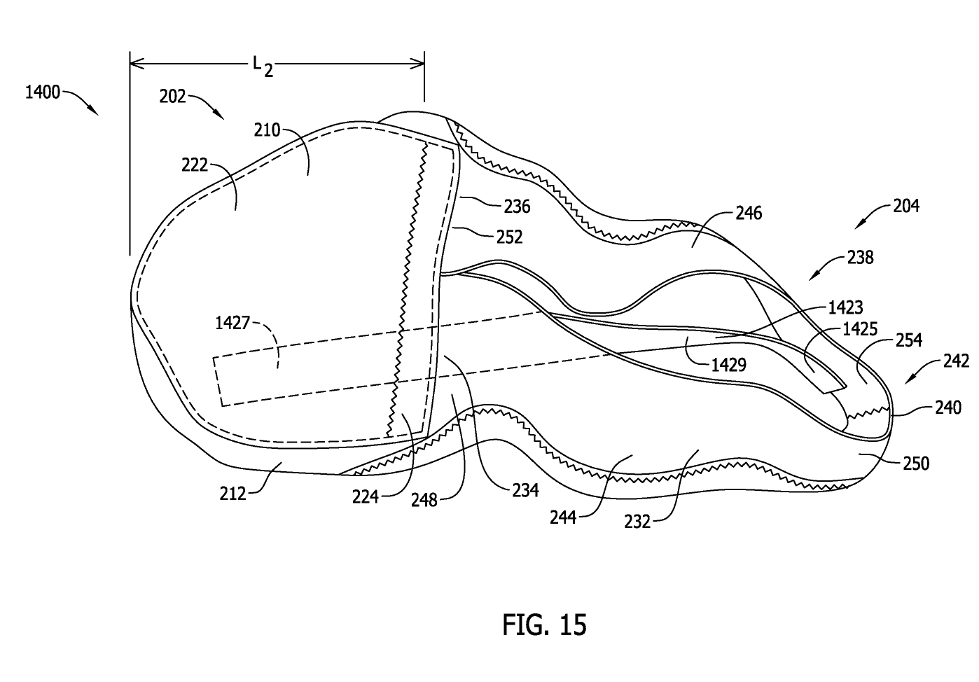

FIG. 14 is a perspective view of a seventh example embodiment of a banded support system 1400 configured to induce the windlass mechanism in the foot, as shown in FIGS. 1A and 1B. FIG. 15 is a bottom view of the banded support system shown in FIG. 14. FIGS. 14 and 15 depict various views of a banded support system 1400 configured to dynamically induce windlass mechanism 102 in foot 100 (both shown in FIG. 1). More particularly, banded support system 1400 is a mobile apparatus configured to induce windlass mechanism 102 substantially constantly during wear or use, providing resting compression of foot 100 to lift mid-foot 108 and shorten plantar fascia 106. “Mobile,” as used herein, refers to an apparatus configured to be with or on a person regardless of location and/or position, in contrast to an apparatus limited in application to particular locations and/or positions. “Resting compression,” as used herein, refers to an application of compression without necessitating action or movement of the person wearing banded support system 1400. For example, “resting compression” occurs while a user of banded support system 1400 is sitting (i.e., no active motion applied to foot 100 by the user).

In the illustrated embodiment, banded support system 1400 includes a forefoot portion 202 and a band 204. In one embodiment, forefoot portion 202 and band 204 are integrally formed as one unitary piece. In such embodiments, band 204 is integrated into forefoot portion 202 during fabrication thereof, for example, is woven into the weave of forefoot portion 202 or are integrally coupled to the weave of forefoot portion 202. Alternatively, forefoot portion 202 and band 204 are distinct, separate components coupled together.

Forefoot portion 202 is configured to surround and/or enclose at least a portion of forefoot 104 of foot 100, in the manner of a typical sock, when banded support system 200 is placed on foot 100. Forefoot portion 202 may enclose one toe (e.g., first or great toe 114), less than all of the toes (e.g., great toe 114 and second toe 116), or all of the toes, corresponding respective metatarsal head(s) 124, and/or at least a portion of mid-foot 108 (all shown in FIGS. 1A and 1B).

Forefoot portion 202 includes a bottom panel 210, an intermediate panel 212, a top panel 214, and a divider panel 1415. Bottom panel 210, intermediate panel 212, top panel 214, and divider panel 1415 are separate components coupled together. Alternatively, bottom panel 210, intermediate panel 212, top panel 214, and/or divider panel 1415 are integrally formed, for example, woven together. Bottom panel 210, intermediate panel 212, and top panel 214 form a cavity 216 for receiving, surrounding, and/or enclosing the portion of forefoot 104, described above, therein. Divider panel 1415 is coupled to bottom panel 210 and divides cavity 216 into an upper cavity 1417 and a lower cavity 1419. Upper cavity 1417 is defined between top panel 214 and divider panel 1415 and is configured to receive the user's toes. Lower cavity 1419 is defined between divider panel 1415 and bottom panel 210 and is configured to receive an orthopedic device therein. For example, and without limitation, lower cavity 1419 may receive a cushion or a platform intended to elevate or angle a portion of forefoot 104.

In the example embodiment, top panel 214 and divider panel 1415 are fabricated from a non-elastic material, bottom panel 210 is fabricated from a semi-elastic material, and intermediate panel 212 is fabricated from an elastic material. Alternatively, bottom panel 210 is fabricated from a non-elastic material. “Non-elastic,” “semi-elastic,” and “elastic” are used herein as relative terms. In other words, an “elastic” material is more elastic and/or exhibits more elastic properties than a “semi-elastic” material. A semi-elastic material is more elastic and/or exhibits more elastic properties than a “non-elastic” material. Additionally, “non-elastic” refers generally to materials that have little or no “give”, “stretch,”, or “expansion” such that they retain their size even under tensile or expansion forces. Elastic materials include, for example, elastic nylon. Semi-elastic materials include, for example, neoprene. Non-elastic materials include, for example, non-elastic nylon. Moreover, at least one of bottom panel 210, intermediate panel 212, top panel 214, and/or divider panel 1415 is fabricated from a material with a relatively high coefficient of static friction on at least one face thereof, or a “tacky” material. For example, bottom panel 210 and/or divider panel 1415 may be fabricated from a tacky material with a tacky top face, such that forefoot 104 experiences less slippage or movement within forefoot portion 202. Additionally, bottom panel 210 may be fabricated from a cushioning material having a thickness and softness such that a cushioning of forefoot 104 is provided. In one embodiment, bottom panel 210 is fabricated from a semi-elastic, tacky, cushioning material, such as a tacky neoprene.

In the example embodiment, the relative elasticity between the panels 210, 212, 214 of forefoot portion 202 causes or encourages a user's toes to be urged forward in forefoot portion 204. Accordingly, one or more of the first through fifth toes are anchored or retained in intermediate panel 212, the most elastic panel. This retention of the toes helps to ensure that the toes remain in the proper placement for activation of windlass mechanism 102 without the need for internal structure in forefoot portion 102, which may be less comfortable for some users. Retention of the toes in intermediate panel 212 may additionally facilitate inducement of dorsiflexion of great toe 114.

Bottom panel 210 includes an elliptical portion 222 with a substantially semi-elliptical shape, corresponding to an approximate outline of forefoot 104. In some embodiment, bottom panel 210 further includes an extension portion 224 extending from elliptical portion 222. Extension portion 224 provides additional surface area of bottom panel 210 for coupling band 204 to bottom panel 210 and/or for increased user comfort. Top panel 214 has a shape that is substantially complementary to the shape of bottom panel 210. More particularly, top panel 214 includes an elliptical portion 226 with a substantially semi-elliptical shape, corresponding to an approximate outline of forefoot 104. Top panel 214 further includes an arcuate edge 1421 that defines a minimum length L1 od top panel 214 as being shorted than a minimum length L2 of bottom panel 210. In the example embodiment, intermediate panel 212 has a substantially rectangular shape. Intermediate panel 212 is coupled to a perimeter of elliptical portion 222 of bottom panel 210 and a perimeter of elliptical portion 226 of top panel 214. Intermediate panel 212 forms an intermediate wall or interface extending from bottom panel 210 to top panel 214.

Band 204 is coupled to forefoot portion 202. In the example embodiment, band 204 is substantially U-shaped and is configured to engage heel 110 (shown in FIGS. 1A and 1B) of foot 100 when banded support system 1400 is placed on foot 100. More particularly, band 204 is configured to curve around heel 110 from a medial side thereof to a lateral side thereof to fully engage heel 110 when banded support system 1400 is worn. As used herein, “U-shaped” refers generally to an approximate U-shape with two “legs” and a rounded or curved midpoint between the two legs. “Substantially U-shaped” or “U-shaped” should be also understood to include shapes with less rounded midpoint, such as an approximate V-shape. In one embodiment, band 204 includes a single band 204. In another embodiment, band 204 includes two bands 204, for example, to provide additional tensile or elastic force about heel 110 to ensure retention of band 204 around heel 110. It should be understood that where general reference to “band 204” is made herein, such description may refer to a one-band embodiment and/or to a two-band embodiment.

Band 204 is fabricated from elastic material(s) exhibiting sufficient elasticity to induce windlass mechanism 102 in foot 100. Accordingly, different banded support systems 200 may include bands 204 with different elastic characteristics, such as a stronger elastic band 204 (i.e., having a higher spring constant) for users requiring more elastic force to induce windlass mechanism 102, or a less elastic band 204 (i.e., having a lower spring constant) for users requiring less force to induce windlass mechanism 102. Therefore, band 204 provides a high level of control of the elastic force imposed on foot 100 while maintaining the comfort of the user of banded support system 1400.

Band 204 includes a first surface 230, also referred to as a “foot-facing”, and an opposing second surface 232. Band 204 further includes a first end 234 and a second end 236. In the example embodiment, first end 234 and second end 236 are coupled to forefoot portion 202. More specifically, first end 234 and second end 236 are coupled to and extend from bottom panel 210 of forefoot portion 202. First end 234 and second end 236 are integrally formed. Alternatively, first end 234 and second end 236 are separate, distinct ends of band 204. First end 234 and second end 236 are positioned proximate one another, in the example embodiment. The relative positions of first end 234 and second end 236 define the U-shape of band 204. Additionally, first end 234 and second end 236 are coupled to forefoot portion 202 in a first, “horizontal” orientation. In the first orientation, first surface 230 of first and second ends 234, 236 is facing “upwards” or towards foot 100 (when worn), and second surface 232 is coupled against bottom panel 210. In an alternative embodiment, in the first orientation, first surface 230 of first and second ends 234, 236 is facing “upwards” and is coupled against a bottom face 220 of bottom panel 210, and second surface 232 of first and second ends 234, 236 is facing “downwards.”

Band 204 further includes a body 238 extending between first end 234 and second end 236. Body 238 includes a midpoint 240 between first end 234 and second end 236. In the illustrated embodiment, midpoint 240 is in a second, “vertical” orientation that is substantially perpendicular to the first orientation. Midpoint 240 forms a U-shaped heel portion 242 configured to receive heel 110. Extending body 238 to form heel portion provides an anchor for primary band 204 and also facilitates establishing an optimal or predetermined amount of force across body 238 of band 204. In other words, extending body 238 tighter through heel portion 242 induces a greater amount of force across body 238 than extending body 238 more loosely through heel portion 242.

Body 238 also includes a first portion 244 extending between first end 234 and midpoint 240 and a second portion 246 extending between second end 236 and midpoint 240. First portion 244 and second portion 246 are accordingly substantially equal in length. First portion 244 includes a first end 248 corresponding to first end 234 of band 204 and a second end 250 positioned at midpoint 240. Second portion 246 includes a first end 252 corresponding to second end 236 of band 204 and a second end 254 positioned at midpoint 240. As such, first portion first end 248 is positioned adjacent second portion first end 252, and first portion second end 250 is positioned adjacent second portion second end 254. In one embodiment, second end 250 of first portion 244 and second end 254 of second portion 246 are separate and distinct. Second end 250 of first portion 244 and second end 254 of second portion 246 are coupled together in the second orientation at midpoint 240, thereby defining midpoint 240 at the intersection thereof. Alternatively, second end 250 of first portion 244 and second end 254 of second portion 246 are integrally formed. In the illustrated embodiment, first portion 244 is substantially parallel with and/or complementary to second portion 246, such that first portion 244 and second portion 246 transition from the first orientation at first ends 248, 252 to the second orientation at second ends 250, 254 in substantially the same manner.

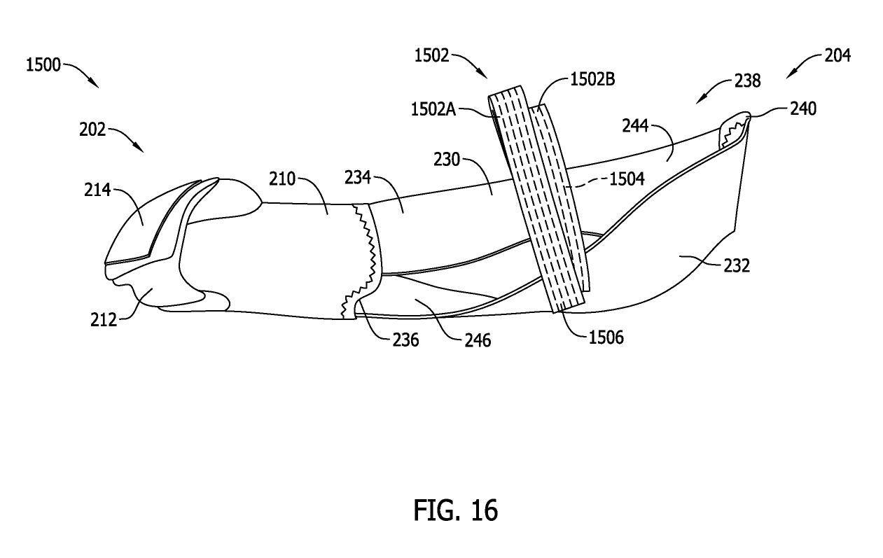

As shown in FIGS. 15 and 16, banded support system 1400 also includes a tensile band 1423 coupled to and extending between band 204 and forefoot portion 202. Specifically, tensile band 1423 includes a first end 1425 coupled to second portion 246 and a second end 1427 coupled to bottom panel 210. More specifically, first end 1425 is coupled to second portion 246 proximate second end 254 in the first orientation such that an inner surface 1429 of tensile band 1423 contacts the user's heel 110. Furthermore, tensile band 1423 extends into lower cavity 1419 such that second end 1427 is coupled to bottom panel 210 proximate a location of the user's first toe 114 when in use. In operation, tensile band 1423 provides additional tensile or elastic force about heel 110 to ensure retention of band 204 around heel 110 and also to provide additional compressive force between band 204 and forefoot portion 202.

FIG. 16 is a perspective view of an eighth example embodiment of a banded support system 1500 configured to induce the windlass mechanism in the foot, as shown in FIGS. 1A and 1B. Banded support system 1500 is substantially similar to banded support system 200, and, as described herein, like components are labeled with like reference numerals.

Forefoot portion 202 is configured to surround and/or enclose at least a portion of forefoot 104 of foot 100 when banded support system 1500 is placed on foot 100. In the embodiment shown in FIG. 16, forefoot portion 202 encloses only one toe (e.g., first or great toe 114) of foot 100.

Banded support system 1500 includes a support band 1502 coupled to and extending between first portion 244 of band 204 and second portion 246 of band 204. Support band 1502 is configured to extend over a user's mid-foot 108 to prevent band 204 from slipping from heel 110 of the user's foot 100. Support band 1502 includes a first end 1504 coupled to first portion 244 and a second end 1506 coupled to second portion 246. More specifically, first end 1504 is coupled to second surface 232 of first portion 244 and second end 1506 is also coupled to second surface 232 of second portion 246. Alternatively, ends 1504 and 1506 may be coupled to first surface 230 of portions 244 and 246. Furthermore, support band 1502 may include a first support band 1502A and a second support band 1502B that may exert the same or different amounts of force on mid-foot 108 to prevent band 204 from slipping.

Support band 1502 is configured to promote and maintain the configuration of body 238 of band 204. Accordingly, support band 1502 prevents body 238 of band 204 from slipping off of heel 110 of a user's foot 100 when in use. Although support band 1502 is shown only in FIG. 16 as for use with single-toe banded support system 1500, support band 1502 may be used with any other embodiment of the banded support systems described herein.

It should be understood that the “securing,” “coupling,” “attaching”, and/or other joining described herein may be accomplished using any suitable securing element(s), such as stitches, weaves, hook-and-loop fasteners, adhesives, and/or any combination(s) thereof.

The above-described banded support system provides an efficient method for inducing the windlass mechanism in a foot. Specifically, the above-described banded support system is configured such that with dorsiflexion of the great toe of the foot, as described herein, the arch or mid-foot rises and the plantar fascia shortens. Moreover, the banded support system is configured to be accessible and mobile for any user thereof, and provides resting compression of the mid-foot. Accordingly, the banded support system may reduce foot stress and provide treatment for or relief from chronic foot pain caused by certain foot conditions such as plantar fasciitis or arthritis. The banded support system described herein is fabricated from materials that enable compression of the foot on which it is being worn, thereby providing substantially constant and passive support to the foot while maintaining the comfort of the user. User comfort is an important aspect of any therapeutic system, such as the banded support system, as increased comfort may increase tolerability and therefore usage of the therapeutic system. For example, the above described embodiments of a banded support system may exist as shown in the figures, be integrally woven and incorporated into a substantially conventional-looking sock, or be incorporated into a shoe. The banded support system described herein provides more comfortable arch or mid-foot support for those with chronic pain. Moreover, the banded support system is configured to actively promote and induce the windlass mechanism for users involved in activities such as running, extended walking, or jumping, which may improve their comfort during such activities and/or may prevent foot injuries caused thereby.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to disclose the embodiments, including the best mode, and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A banded support system configured to induce a windlass mechanism in a foot, said banded support system comprising:
a forefoot portion configured to enclose at least a portion of a forefoot of the foot; and
a band coupled to said forefoot portion, said band comprising:
a first end extending from said forefoot portion in a first orientation;
a second end extending from said forefoot portion in the first orientation, wherein said first end is positioned proximate said second end such that said band is substantially U-shaped; and
a midpoint positioned between said first and second ends in a second orientation perpendicular to the first orientation.

2. A banded support system in accordance with claim 1, wherein said band further comprises:
a first portion comprising a first end corresponding to said band first end and a distal second end positioned at said band midpoint; and
a second portion comprising a first end corresponding to said band second end and a distal second end positioned at said band midpoint, wherein said first portion second end and said second portion second end are coupled together at said band midpoint.

3. A banded support system in accordance with claim 1, wherein said forefoot portion comprises a bottom panel, an intermediate panel, and a top panel, wherein said bottom panel, said intermediate panel, and said top panel are coupled together to form a cavity configured to receive the at least a portion of the forefoot.

4. A banded support system in accordance with claim 3, wherein said top panel is fabricated from a non-elastic material, said bottom panel is fabricated from a semi-elastic material that is more elastic than the non-elastic material, and said intermediate panel is fabricated from an elastic material that is more elastic than the semi-elastic material.

5. A banded support system in accordance with claim 3, further comprising a divider panel coupled to one of said bottom panel, said intermediate panel, and said top panel, wherein said divider panel is configured to divide the cavity into an upper cavity configured to receive a user's toes and a lower cavity configured to receive an orthopedic device.

6. A banded support system in accordance with claim 3, wherein the cavity is configured to receive a user's toes, and wherein a relative elasticity of the non-elastic material of said top panel and the semi-elastic material of said bottom panel causes the user's toes to be retained in said intermediate panel of said forefoot portion.

7. A support system in accordance with claim 3, wherein the cavity is configured to receive less than all of a user's toes.

8. A banded support system in accordance with claim 3, wherein said band first end and said band second end are coupled to said bottom panel of said forefoot portion.

9. A banded support system in accordance with claim 1, wherein said band comprises two bands, each band of said two bands comprising a respective first end and a respective second end.

10. A banded support system in accordance with claim 1, wherein said band first end and said band second end are integrally formed.

11. A banded support system in accordance with claim 1, wherein said band is integral to said forefoot portion.

12. A banded support system in accordance with claim 1, wherein said band and said forefoot portion are separate components coupled together.

13. A banded support system in accordance with claim 1, wherein said band is fabricated from an elastic material.

14. A banded support system configured to induce a windlass mechanism in a foot, said banded support system comprising:
a forefoot portion configured to enclose at least a portion of a forefoot of the foot; and
a band coupled to said forefoot portion, said band comprising:
a first portion comprising a first end and a second end, said first portion first end extending from said forefoot portion in a first orientation, said first portion terminating at said first portion second end in a second orientation perpendicular to the first orientation; and
a second portion comprising a first end and a second end, said second portion first end extending from said forefoot portion in the first orientation, said second portion terminating at said second portion second end in the second orientation, wherein said second portion second end is coupled to said first portion second end.

15. A banded support system in accordance with claim 14, wherein the banded support system is incorporated into a sock.

16. A banded support system in accordance with claim 14, further comprising a support band coupled to and extending between said first portion and said second portion, wherein said support band is configured to extend over a user's foot to prevent said band from slipping from a heel portion of the user's foot.

17. A banded support system in accordance with claim 14, further comprising a tensile band coupled to and extending between said forefoot portion and said band.

18. A banded support system in accordance with claim 17, wherein said tensile band is coupled to said band proximate one of said first portion second end or said second portion second end.

19. A banded support system in accordance with claim 1, wherein said forefoot portion comprises a bottom panel, an intermediate panel, and a top panel, wherein said bottom panel, said intermediate panel, and said top panel are coupled together to form a cavity configured to receive the at least a portion of the forefoot.

20. A banded support system in accordance with claim 6, wherein said top panel is fabricated from a non-elastic material, said bottom panel is fabricated from a semi-elastic material that is more elastic than the non-elastic material, and said intermediate panel is fabricated from an elastic material that is more elastic than the semi-elastic material.
